(19) United States
(12) Patent Application Publication
(10) Pub. No.: US 2006/0178624 A1
(43) Pub. Date: Aug. 10, 2006

(54) Retractable safety syringe with push rod in plunger

(76) Inventor: Chao-Song Hsue, Taichung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/168,519

(22) Filed: Jun. 29, 2005

(30) Foreign Application Priority Data
Apr. 11, 2005 (TW) 94111387
Jul. 2, 2004 (TW) 93120078

(51) Int. Cl. A61M 5/00; A61M 5/32

(52) U.S. Cl. 604/110; 604/192

(57) ABSTRACT

A safety syringe includes a barrel and a plunger is axially and movably inserted in the barrel. The plunger has a stopper connected to a first end thereof which is inserted in the barrel and a through hole is defined through the first end of the plunger. A push rod is axially and movably inserted into the plunger and an engaging member is connected to a first end of the push rod. A second end of the push rod is received in a second end of the plunger. A needle cannula is removably engaged with a first end of the barrel and has an engaging hole defined in an end thereof. The engaging member extends through the through hole and is engaged with the engaging hole of the needle cannula after injection by pushing the push rod toward the needle cannula. The needle cannular is then pulled backward and received in the barrel by pulling the plunger.

### FIELD OF THE INVENTION

The present invention relates to a safety syringe wherein a push rod is received in the plunger and the needle cannula is pulled in the barrel after use to protect the users.

### BACKGROUND OF THE INVENTION

A conventional safety syringe is disclosed in FIG. 13 and generally includes a barrel 6 with a plunger 8 movably received in a passage in the barrel 6, a connection member 61 received in the barrel 6 and a needle cannula 7 is connected to the connection member 61 and extends through an end of the barrel 6. The plunger 8 has an engaging member 81 connected to an end thereof and the connection member 61 has a recess 611 for receiving the engaging member 81 when the plunger 8 is pushed into the barrel 6 to eject medicine in the barrel 6 out from the needle cannula 7. The needle cannular 7 is pulled back into the barrel 6 with the plunger 8 after use so that users are not hurt by the needle cannula 7. However, the plunger 8 has one end extending out from the barrel 6 before use and the plunger 8 might be pushed unintentionally by the users or during transportation. Once the plunger 8 is pushed to the bottom barrel 6, and is connected with the connection member 61, they cannot be separated and the syringe has to be discarded.

Furthermore, as shown in FIG. 14, for a larger volume of syringe, the locking tip 91 is located eccentrically on an end of the barrel 9 and the needle cannula 92 is connected to the locking tip 91. If the same safety mechanism is used for this type of syringe, the engaging member has to be located eccentrically on the plunger and is in alignment with the eccentric needle cannula 92. This makes the connection of the engaging member and the needle cannula 92 to be more difficult.

The present invention intends to provide a safety syringe wherein a push rod is received in the plunger and does not extend beyond the plunger so that the needle cannula is not connected to the engaging member of the push rod except the user intends to. Besides, the syringe of the present invention may use with a standard Luer needle cannula without extra parts and amendments. The engaging hole of the Luer needle cannula can be directly connected to the neck of the push rod, or a cover with an engaging hole can be connected to the Luer needle cannula to be connected with the push rod.

### SUMMARY OF THE INVENTION

The present invention relates to a safety syringe which comprises a barrel having a first passage so that a plunger is axially and movably inserted in the first passage. The plunger has a stopper connected to a first end thereof and inserted in the first passage. A through hole is defined through the first end of the plunger. A push rod is axially and movably inserted into the plunger and an engaging member with a connection portion is connected to a first end of the push rod. A second end of the push rod is received in a second end of the plunger. A needle cannula is engaged with a first end of the barrel and has an engaging hole defined in an end thereof. The engaging member extends through the through hole and is engaged with the engaging hole to combine the push rod and the needle cannula after injection, and the needle cannula is pulled backward and received in the barrel by pulling the plunger backward.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view to show the safety syringe of the present invention;

FIG. 2 is a cross sectional view to show the safety syringe of the present invention;

FIG. 3 is an enlarged cross sectional view to show that the engaging member is about to extend through the through hole and to be engaged with the needle cannula;

FIG. 4 is a cross sectional view to show that the engaging member extends through the through hole and is engaged with the needle cannula by pushing the push rod;

FIG. 5 shows an embodiment of the engaging hole in the safety syringe of the present invention;

FIG. 6 shows another embodiment of the engaging hole in the safety syringe of the present invention;

FIG. 7 shows that the needle cannula is pulled back into the barrel;

FIG. 8 is a cross sectional view to show another embodiment of the safety syringe of the present invention;

FIG. 9 shows a top view of the safety syringe of the embodiment in FIG. 8;

FIG. 10 shows a bottom view of the safety syringe of the embodiment in FIG. 8;

FIG. 11 shows a cross sectional view of yet another embodiment of the safety syringe of the present invention;

FIG. 12 shows the connection of the needle cannula and the push rod;

FIG. 13 shows an embodiment of a conventional syringe, and

FIG. 14 is another embodiment of a conventional syringe.

### DETAILED DESCRIPTION OF THE PREFERRED EMBODIMENT

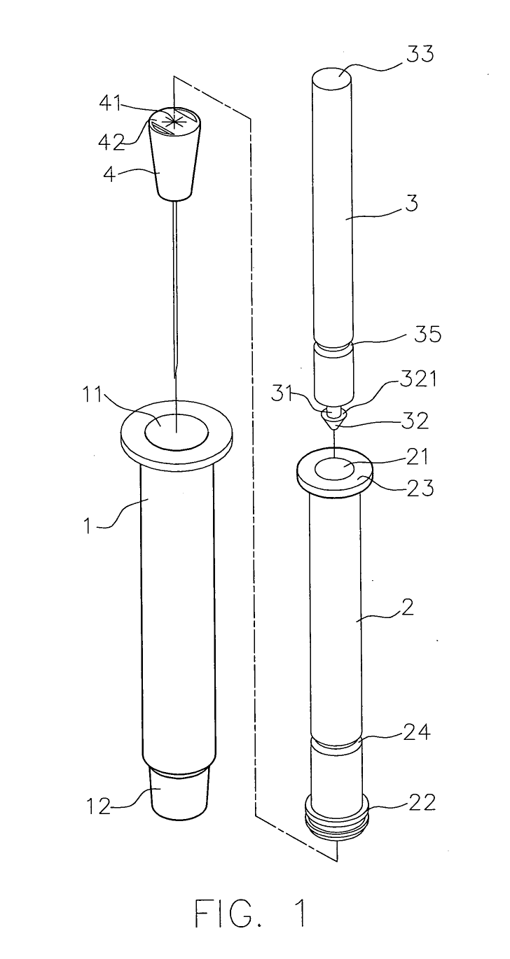

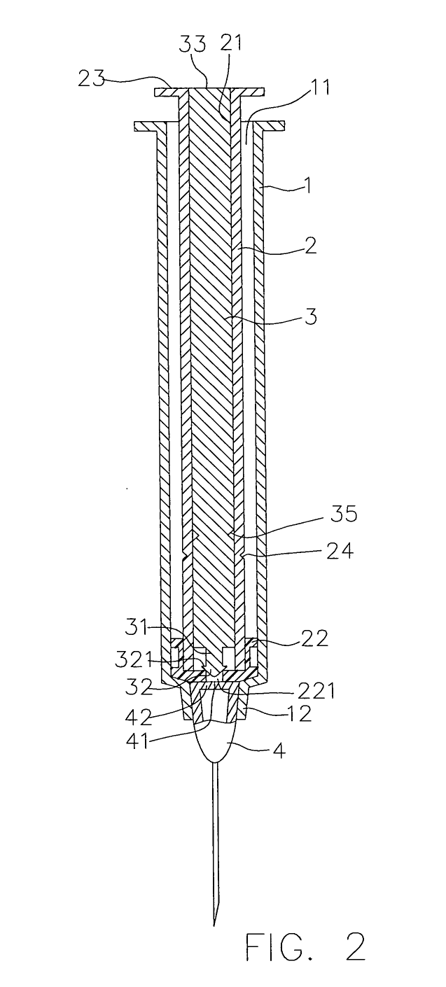

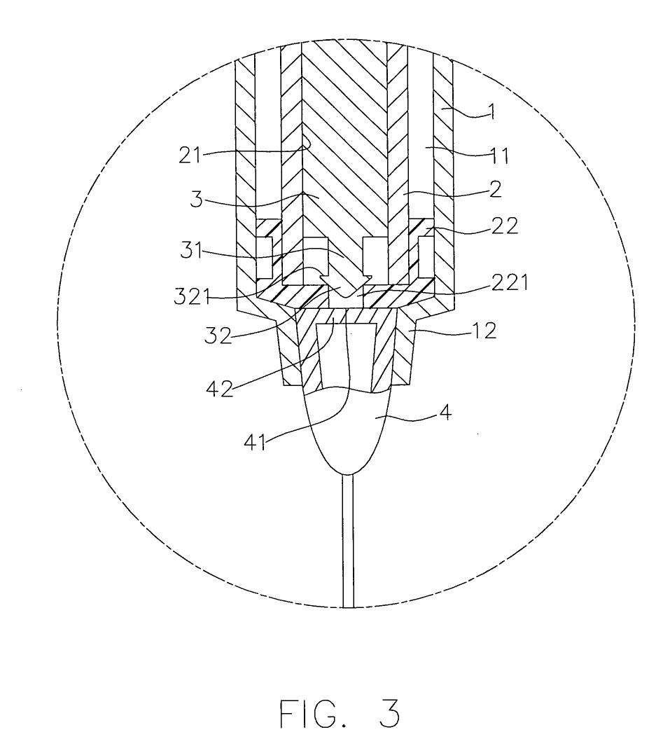

Referring to FIGS. 1 to 3, the safety syringe of the present invention comprises a barrel 1 having a first passage 11 defined therein and an extension 12 extends from a first end of the barrel 1 so that a needle cannula 4 is removably extended through the extension 12. A finger flange extends outward from a second end of the barrel 1. A plunger 2 has a first end thereof axially and movably inserted in the first passage 11 of the barrel 1 and the plunger 2 has a stopper 22 connected to a first end thereof which is inserted in the first passage 11. A through hole 221 is defined through the first end of the plunger 2. A thumb rest 23 extends outward from a second end of the plunger 2. A second passage 21 is defined in the plunger 2. A first groove 24 is defined in the outer periphery of the plunger 2.

A push rod 3 has a first end thereof axially and movably inserted into the second passage 21 in the plunger 2 and an engaging member 32 is connected to the first end of the push rod 3. The engaging member 32 has a cone-shaped connection portion 321 and a narrow neck 31 is connected between the first end of the push rod 3 and the engaging member 32. The neck 31 has a smaller diameter than that of the engaging member 32 so as to form the cone or arrow-head connection portion 321. A second end 33 of the push rod 3 is received into a second end of the plunger 2 or in flush with the second end of the plunger 2. In this embodiment, the second end 33 of the push rod 3 in flush with the second end of the plunger 2, this arrangement of the second end of the push rod 3 is for description purpose only and is not limited to be in flush with the second end of the plunger 2.

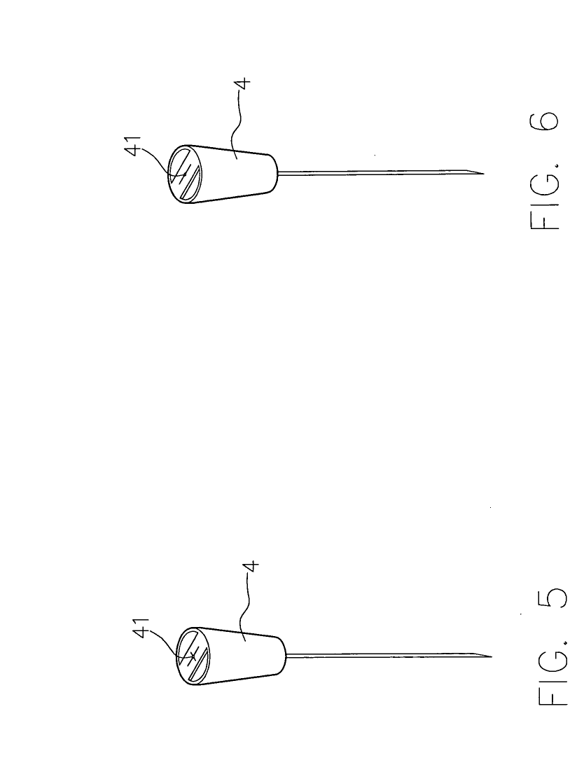

The needle cannula 4 includes a cover 42 through which an engaging hole 41 is defined which is defined by multiple slits. The engaging hole 41 can also be made by a cruciform slit as shown in FIG. 5 or even a straight slit as shown in FIG. 6.

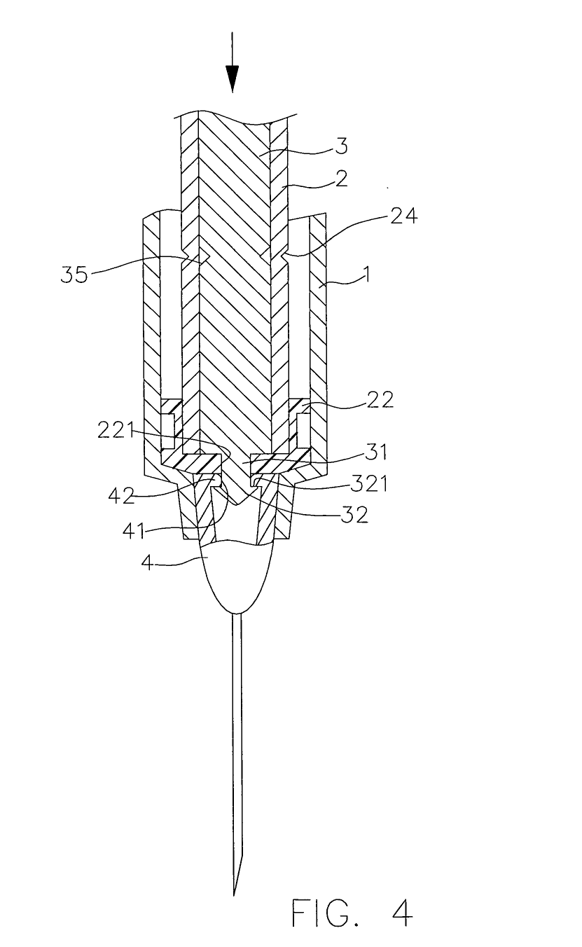

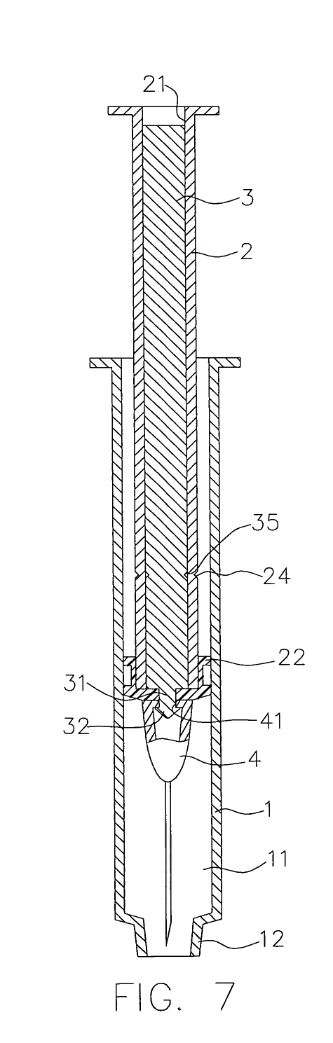

As shown in FIGS. 4 and 7, after injection, the plunger 2 is completely pushed into the barrel 1 and the user pushes the push rod 3 toward the needle cannula 4 to let the engaging member 32 extend through the through hole 221 and be engaged with the engaging hole 41. The connection portion 321 of the engaging member 32 is then hooked with the cover 42 to combine the push rod 3 with the needle cannula 4. The user then pulls the plunger backward and the needle cannula 4 is pulled with the plunger 2 and received in the barrel 1 as shown in FIG. 7. The user bends the plunger 2 together with the push rod 3 to break the plunger 2 together with the push rod 3 at the first and second grooves 24, 35. By this way, the needle cannula 4 is permanently received in the barrel 1.

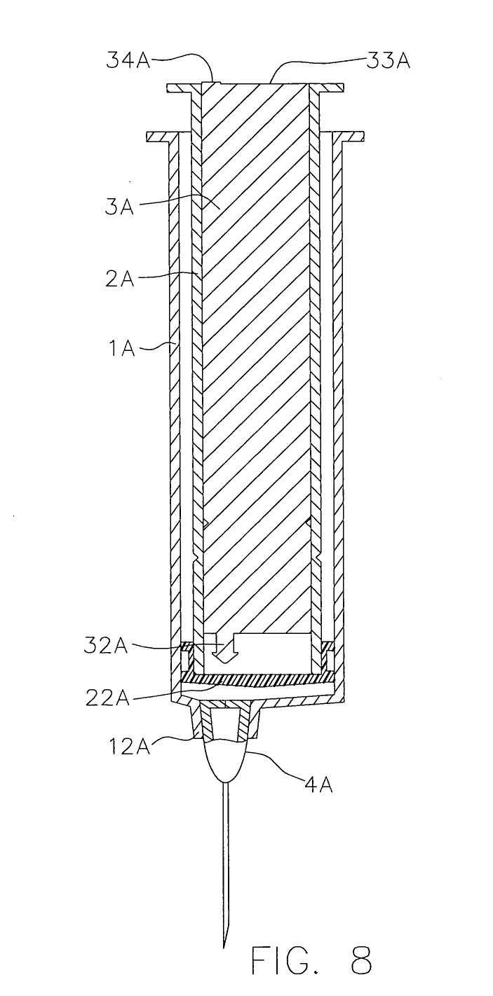

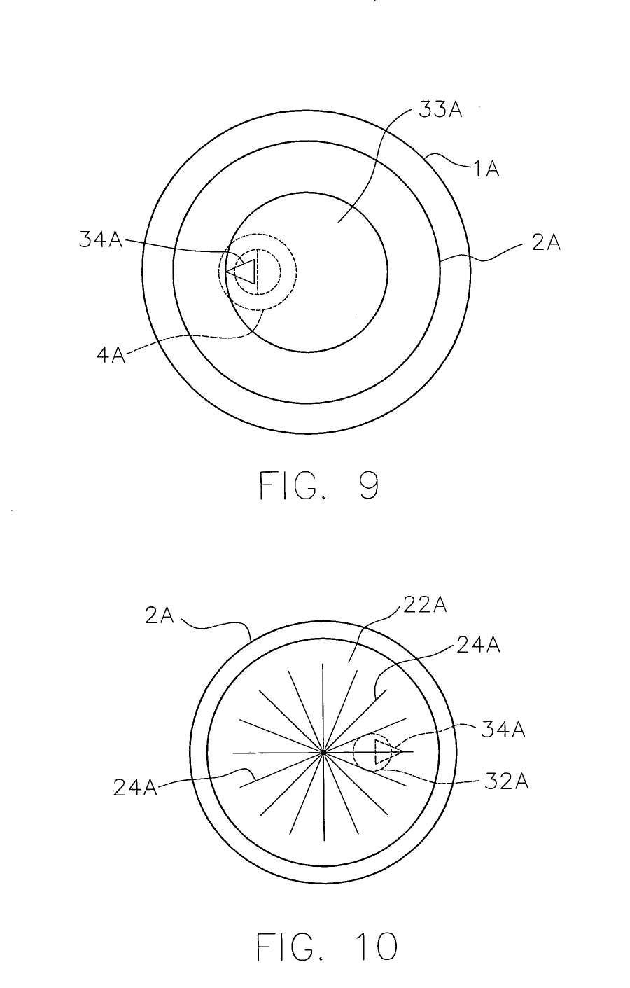

FIGS. 8 to 10 show another embodiment of the present invention wherein the extension 12A is located eccentrically of the first end of the barrel 1A and the engaging member 32A is located eccentrically on the first end of the push rod 3A which is movably inserted in the plunger 2A. The needle cannula 4A is engaged with the extension 12A. The through hole 221A in the stopper 22A of the plunger 2A is defined by a star-shaped slit comprising multiple slits 24A. A mark 34A is connected on the second end 33A of the push rod 3A and in alignment with the eccentric engaging member 32A so that the user can easily push the push rod 3A and extend the engaging member 32A through the through hole 221A and connected with the needle cannula 4A.

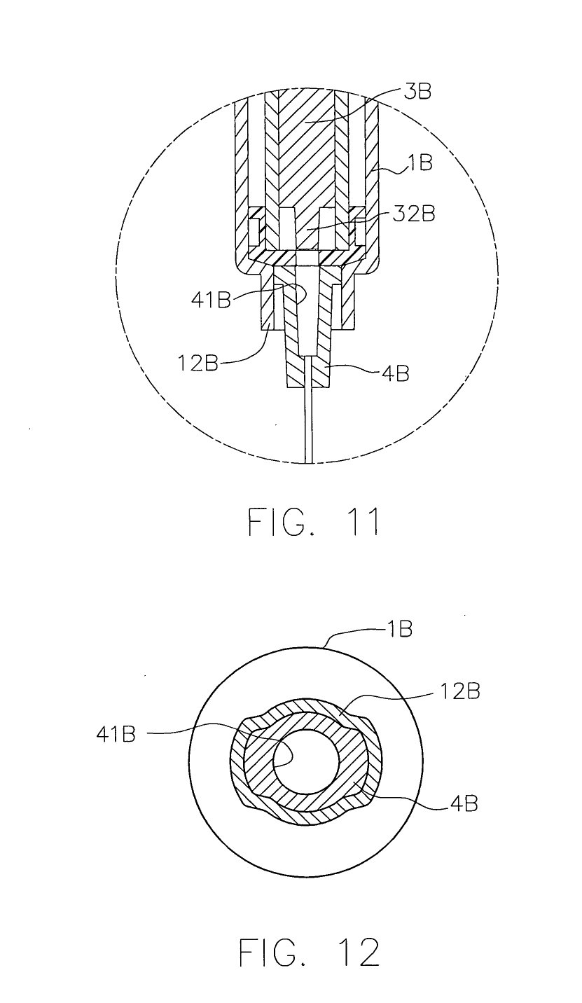

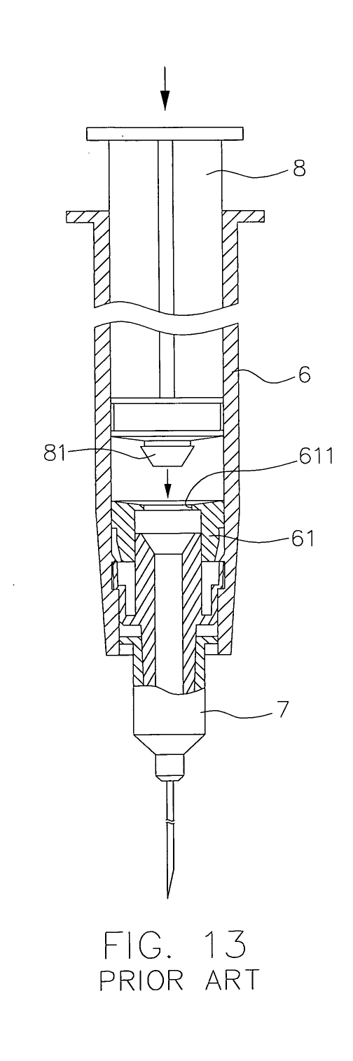

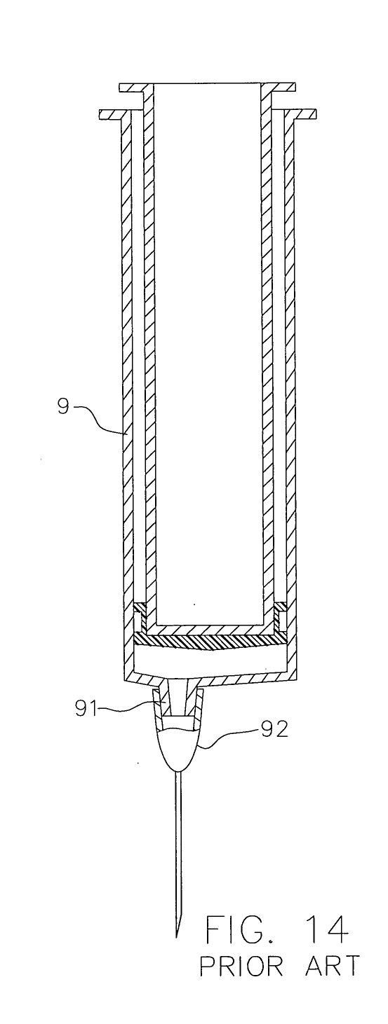

FIGS. 11 and 12 show yet another embodiment of the safety syringe of the present invention, wherein the needle cannula 4B is a standard Luer needle cannula and includes an engaging hole 41B. The extension 12B of the barrel 1B includes an inner periphery that fits the Luer needle cannula 4B so as to securely connect the Luer needle cannula 4B. The engaging member 32B of the push rod 3B is a cone-shaped member which is securely engaged with the engaging hole 41B of the needle cannula 4B so as to be connected to the needle cannula 4B and to pull the needle cannula 4B in the barrel 1B.

While we have shown and described the embodiment in accordance with the present invention, it should be clear to those skilled in the art that further embodiments may be made without departing from the scope of the present invention.

What is claimed is:

1. A retractable safety syringe with push rod in plunger comprising:
a barrel having a first passage defined therein and a plunger axially and movably inserted in the first passage of the barrel, the plunger having a stopper connected to a first end thereof which is inserted in the first passage, a through hole defined through the first end of the plunger;
a push rod axially and movably inserted into the plunger and an engaging member connected to a first end of the push rod, a second end of the push rod received in a second end of the plunger, and
a needle cannula removably engaged with a first end of the barrel and having an engaging hole defined in an end thereof, the engaging member extending through the through hole and being shaped so as to be engaged with the engaging hole after the syringe is used.

2. The syringe as claimed in claim 1, wherein the barrel includes an extension extending from the first end thereof and the needle cannula is securely extended through the extension.

3. The syringe as claimed in claim 1, wherein the second end of the push rod is in flush with the second end of the plunger.

4. The syringe as claimed in claim 1, wherein a neck is connected between the first end of the push rod and the engaging member, the neck has a smaller diameter than that of the engaging member.

5. The syringe as claimed in claim 1, wherein the needle cannula includes a cover through which the engaging hole is defined.

6. The syringe as claimed in claim 1, wherein the needle cannula includes an engaging hole.

7. The syringe as claimed in claim 5 or 6, wherein engaging hole is defined by a star-shaped slit with which the engaging member is engaged.

8. The syringe as claimed in claim 5 or 6, wherein engaging hole is defined by a cruciform slit with which the engaging member is engaged.

9. The syringe as claimed in claim 5 or 6, wherein engaging hole is defined by a straight slit with which the engaging member is engaged.

10. The syringe as claimed in claim 1, wherein the through hole in the stopper of the plunger is defined by multiple slits through which the engaging member is capable to extend.

11. The syringe as claimed in claim 1, wherein the extension is located eccentrically of the first end of the barrel so as to be connected with the needle cannula, the engaging member is connected eccentrically on the first end of the push rod in the plunger.

12. The syringe as claimed in claim 1, wherein a mark is connected on the second end of the push rod and in alignment with the engaging member of the push rod.

13. The syringe as claimed in claim 1, wherein the plunger includes a first groove defined in the outer periphery thereof and the push rod has a second groove defined in an outer periphery thereof.

14. The syringe as claimed in claim 1, wherein the needle cannula is a standard Luer needle cannula and an inner periphery of the extension on the barrel is securely fitted with the standard Luer needle cannula, the engaging member of the push rod is a cone-shaped member which is securely engaged with the engaging hole of the standard Luer needle cannula.

15. The syringe as claimed in claim 1, wherein the engaging member has a connection portion in radial direction so as to be engaged with the engaging hole of the needle cannula.
